(19) United States
(12) Patent Application Publication
(10) Pub. No.: US 2018/0360786 A1
(43) Pub. Date: Dec. 20, 2018

(54) (R)-CARBIDOPA ALONE OR IN COMBINATION WITH A STEROID FOR TREATING PAIN, INFLAMMATION, AN INFLAMMATORY DISEASE, AN IMMUNE OR AUTOIMMUNE DISEASE AND TUMOUR GROWTH

(71) Applicant: PROXIMAGEN GROUP LIMITED, Cambridge Cambridgeshire (GB)

(72) Inventors: Martyn PRITCHARD, Cambridge (GB); Peter RICHARDSON, Cambridge (GB)

(73) Assignee: PROXIMAGEN GROUP LIMITED, Cambridge Cambridgeshire (GB)

(21) Appl. No.: 15/781,871

(22) PCT Filed: Dec. 7, 2016
(86) PCT No.: PCT/GB2016/053849

§ 371 Date: Jun. 6, 2018

(30) Foreign Application Priority Data
Dec. 7, 2015 (GB) 1521541.1

(51) Int. Cl. A61K 31/198; A61K 31/573

(52) U.S. Cl. CPC A61K 31/198; A61K 31/573

(57) ABSTRACT

The present invention relates to (R)-carbidopa and pharmaceutical compositions thereof, and to a combined preparation of (R)-carbidopa and a steroid, and to the use of the same in the treatment or prevention of medical conditions such as pain, inflammatory diseases, immune or autoimmune disorders and tumour growth.

### FIELD OF THE INVENTION

This invention relates to (R)-carbidopa, and pharmaceutical compositions comprising the same, in the treatment or prevention of medical conditions such as pain, inflammatory diseases, immune or autoimmune disorders and tumour growth. The invention relates also to a combined preparation comprising (R)-carbidopa and a steroid, and the use of the combined preparation in medicine, particularly in the treatment or prevention of medical conditions such as pain, inflammatory diseases, immune or autoimmune disorders and the inhibition of tumour growth.

### BACKGROUND ART

Semicarbazide-sensitive amine oxidase (SSAO), otherwise known as Vascular Adhesion Protein-1 (VAP-1) or Amine Oxidase, Copper Containing 3 (AOC3), belongs to the copper-containing amine oxidase family of enzymes (EC.1.4.3.6). Members of this enzyme family are sensitive to inhibition by semicarbazide and utilize cupric ion and protein-derived topa quinone (TPQ) cofactor in the oxidative deamination of primary amines to aldehydes, hydrogen peroxide, and ammonia according to the following reaction:

R—CH₂—NH₂+O₂→R—CHO+H₂O₂+NH₃

Known substrates for human SSAO include endogenous methylamine and aminoacetone as well as some xenobiotic amines such as benzylamine [Lyles, Int. J. Biochem. Cell Biol. 1996, 28, 259-274; Klinman, Biochim. Biophys. Acta 2003, 1647(1-2), 131-137; Matyus et al., Curr. Med. Chem. 2004, 11(10), 1285-1298; O'Sullivan et al., Neurotoxicology 2004, 25(1-2), 303-315]. In analogy with other copper-containing amine oxidases, DNA-sequence analysis and structure determination suggest that the tissue-bound human SSAO is a homodimeric glycoprotein consisting of two 90-100 kDa subunits anchored to the plasma membrane by a single N-terminal membrane spanning domain [Morris et al., J. Biol. Chem. 1997, 272, 9388-9392; Smith et al., J. Exp. Med. 1998, 188, 17-27; Airenne et al., Protein Science 2005, 14, 1964-1974; Jakobsson et al., Acta Crystallogr. D Biol. Crystallogr. 2005, 61(Pt 11), 1550-1562].

SSAO activity has been found in a variety of tissues including vascular and non-vascular smooth muscle tissue, endothelium, and adipose tissue [Lewinsohn, Braz. J. Med. Biol. Res. 1984, 17, 223-256; Nakos & Gossrau, Folia Histochem. Cytobiol. 1994, 32, 3-10; Yu et al., Biochem. Pharmacol. 1994, 47, 1055-1059; Castillo et al., Neurochem. Int. 1998, 33, 415-423; Lyles & Pino, J. Neural. Transm. Suppl. 1998, 52, 239-250; Jaakkola et al., Am. J. Pathol. 1999, 155, 1953-1965; Morin et al., J. Pharmacol. Exp. Ther. 2001, 297, 563-572; Salmi & Jalkanen, Trends Immunol. 2001, 22, 211-216]. In addition, SSAO protein is found in blood plasma and this soluble form appears to have similar properties as the tissue-bound form [Yu et al., Biochem. Pharmacol. 1994, 47, 1055-1059; Kurkijärvi et al., J. Immunol. 1998, 161, 1549-1557]. It has recently been shown that circulating human and rodent SSAO originates from the tissue-bound form [Gökturk et al., Am. J. Pathol. 2003, 163(5), 1921-1928; Abella et al., Diabetologia 2004, 47(3), 429-438; Stolen et al., Circ. Res. 2004, 95(1), 50-57], whereas in other mammals the plasma/serum SSAO is also encoded by a separate gene called AOC4 [Schwelberger, J. Neural. Transm. 2007, 114(6), 757-762].

The precise physiological role of this abundant enzyme has yet to be fully determined, but it appears that SSAO and its reaction products may have several functions in cell signalling and regulation. For example, recent findings suggest that SSAO plays a role in both GLUT4-mediated glucose uptake [Enrique-Tarancon et al., J. Biol. Chem. 1998, 273, 8025-8032; Morin et al., J. Pharmacol. Exp. Ther. 2001, 297, 563-572] and adipocyte differentiation [Fontana et al., Biochem. J. 2001, 356, 769-777; Mercier et al., Biochem. J. 2001, 358, 335-342]. In addition, SSAO has been shown to be involved in inflammatory processes where it acts as an adhesion protein for leukocytes [Salmi & Jalkanen, Trends Immunol. 2001, 22, 211-216; Salmi & Jalkanen, in “Adhesion Molecules: Functions and Inhibition” K. Ley (Ed.), 2007, pp. 237-251], and might also play a role in connective tissue matrix development and maintenance [Langford et al., Cardiovasc. Toxicol. 2002, 2(2), 141-150; Gokturk et al., Am. J. Pathol. 2003, 163(5), 1921-1928]. Moreover, a link between SSAO and angiogenesis has recently been discovered [Noda et al., FASEB J. 2008, 22(8), 2928-2935], and based on this link it is expected that inhibitors of SSAO have an anti-angiogenic effect.

Several studies in humans have demonstrated that SSAO activity in blood plasma is elevated in conditions such as congestive heart failure, diabetes mellitus, Alzheimer's disease, and inflammation [Lewinsohn, Braz. J. Med. Biol. Res. 1984, 17, 223-256; Boomsma et al., Cardiovasc. Res. 1997, 33, 387-391; Ekblom, Pharmacol. Res. 1998, 37, 87-92; Kurkijärvi et al., J. Immunol. 1998, 161, 1549-1557; Boomsma et al., Diabetologia 1999, 42, 233-237; Meszaros et al., Eur. J. Drug Metab. Pharmacokinet. 1999, 24, 299-302; Yu et al., Biochim. Biophys. Acta 2003, 1647(1-2), 193-199; Matyus et al., Curr. Med. Chem. 2004, 11(10), 1285-1298; O'Sullivan et al., Neurotoxicology 2004, 25(1-2), 303-315; del Mar Hernandez et al., Neurosci. Lett. 2005, 384(1-2), 183-187]. The mechanisms underlying these alterations of enzyme activity are not clear. It has been suggested that reactive aldehydes and hydrogen peroxide produced by endogenous amine oxidases contribute to the progression of cardiovascular diseases, diabetic complications and Alzheimer's disease [Callingham et al., Prog. Brain Res. 1995, 106, 305-321; Ekblom, Pharmacol. Res. 1998, 37, 87-92; Yu et al., Biochim. Biophys. Acta 2003, 1647(1-2), 193-199; Jiang et al., Neuropathol Appl Neurobiol. 2008, 34(2), 194-204]. Furthermore, the enzymatic activity of SSAO is involved in the leukocyte extravasation process at sites of inflammation where SSAO has been shown to be strongly expressed on the vascular endothelium [Salmi et al., Immunity 2001, 14(3), 265-276; Salmi & Jalkanen, in “Adhesion Molecules: Functions and Inhibition” K. Ley (Ed.), 2007, pp. 237-251]. Accordingly, inhibition of SSAO has been suggested to have a therapeutic value in the prevention of diabetic complications and in inflammatory diseases [Ekblom, Pharmacol. Res. 1998, 37, 87-92; Salmi et al., Immunity 2001, 14(3), 265-276; Salter-Cid et al., J. Pharmacol. Exp. Ther. 2005, 315(2), 553-562]. WO2007/146188 teaches that blocking SSAO activity inhibits leucocyte recruitment, reduces the inflammatory response, and is expected to be beneficial in prevention and treatment of seizures, for example, in epilepsy.

O'Rourke et al (J Neural Transm. 2007; 114(6):845-9) examined the potential of SSAO inhibitors in neurological diseases, having previously demonstrated the efficacy of SSAO inhibition in a rat model of stroke. An SSAO inhibitor is tested on relapsing-remitting experimental autoimmune encephalomyelitis (EAE), a mouse model that shares many characteristics with human multiple sclerosis. The data demonstrates the potential clinical benefit of small molecule anti-SSAO therapy in this model and therefore in treatment of human multiple sclerosis.

SSAO knockout animals are phenotypically overtly normal but exhibit a marked decrease in the inflammatory responses evoked in response to various inflammatory stimuli [Stolen et al., Immunity 2005, 22(1), 105-115]. In addition, antagonism of its function in wild type animals in multiple animal models of human disease (e.g. carrageenan-induced paw inflammation, oxazolone-induced colitis, lipopolysaccharide-induced lung inflammation, collagen-induced arthritis, endotoxin-induced uveitis) by the use of antibodies and/or small molecules has been shown to be protective in decreasing the leukocyte infiltration, reducing the severity of the disease phenotype and reducing levels of inflammatory cytokines and chemokines [Kirton et al., Eur. J. Immunol. 2005, 35(11), 3119-3130; Salter-Cid et al., J. Pharmacol. Exp. Ther. 2005, 315(2), 553-562; McDonald et al., Annual Reports in Medicinal Chemistry 2007, 42, 229-243; Salmi & Jalkanen, in “Adhesion Molecules: Functions and Inhibition” K. Ley (Ed.), 2007, pp. 237-251; Noda et al., FASEB J. 2008 22(4), 1094-1103; Noda et al., FASEB J. 2008, 22(8), 2928-2935]. This anti-inflammatory protection seems to be afforded across a wide range of inflammatory models all with independent causative mechanisms, rather than being restricted to one particular disease or disease model. This would suggest that SSAO may be a key nodal point for the regulation of the inflammatory response, and it seems therefore likely that SSAO inhibitors may be effective anti-inflammatory drugs in a wide range of human diseases.

Fibrosis can result from chronic tissue inflammation when the resolution of the inflammation is partly abrogated by the chronic nature of the inflammatory stimulus. The result can be inappropriate repair of the tissue with excessive extracellular matrix deposition (including collagen) with tissue scarring. This is a consequence of myofibroblast activation by stimuli including fibronectin and reactive oxygen species as well as growth factors such as transforming growth factor-β-1 (TGFβ-1), insulin-like growth factor-I (IGF-I), platelet-derived growth factor (PDGF) and connective tissue growth factor (CTGF) resulting in increased production of collagen, elastin, hyaluronan, glycoproteins and proteoglycans. In addition the activity of invading macrophages plays a crucial part in regulating the repair and fibrotic processes.

VAP-1 has also been implicated in the progression and maintenance of fibrotic diseases especially in the liver. Weston and Adams (J Neural Transm. 2011, 118(7), 1055-64) have summarised the experimental data implicating VAP-1 in liver fibrosis. Weston et al (EASL Poster 2010) showed highly increased expression of VAP-1 in human fibrotic liver, particularly associated with the activated myofibroblasts and collagen fibrils. This anatomical association with fibrosis was consistent with the observation that blockade of VAP-1 accelerated the resolution of carbon tetrachloride induced fibrosis, and suggested a role for the VAP-1/SSAO enzyme product H2O2 in the activation of the myofibroblasts. The same authors also showed that the pro-fibrotic growth factor TGFβ increased the expression of VAP-1 in liver cells by approximately 50-fold. In addition VAP-1 has been implicated in inflammation of the lung (e.g. Singh et al., 2003, Virchows Arch 442:491-495) suggesting that VAP-1 blockers would reduce lung inflammation and thus be of benefit to the treatment of cystic fibrosis by treating both the pro-fibrotic and pro-inflammatory aspects of the disease.

SSAO (VAP-1) is up regulated in gastric cancer and has been identified in the tumour vasculature of human melanoma, hepatoma and head and neck tumours (Yoong K F, McNab G, Hubscher S G, Adams D H. (1998), J Immunol 160, 3978-88.; Irjala H, Salmi M, Alanen K, Gre'nman R, Jalkanen S (2001), Immunol. 166, 6937-6943; Forster-Horvath C, Dome B, Paku S, et al. (2004), Melanoma Res. 14, 135-40.). One report (Marttila-lchihara F, Castermans K, Auvinen K, Oude Egbrink M G, Jalkanen S, Griffioen A W, Salmi M. (2010), J Immunol. 184, 3164-3173.) has shown that mice bearing enzymically inactive VAP-1 grow melanomas more slowly, and have reduced tumour blood vessel number and diameter. The reduced growth of these tumours was also reflected in the reduced (by 60-70%) infiltration of myeloid suppressor cells. Encouragingly VAP-1 deficiency had no effect on vessel or lymph formation in normal tissue.

For the above reasons, it is expected that inhibition of SSAO will reduce the levels of pro-inflammatory enzyme products (aldehydes, hydrogen peroxide and ammonia) whilst also decreasing the adhesive capacity of immune cells and correspondingly their activation and final extra-vasation. Diseases where such an activity is expected to be therapeutically beneficial include all diseases where immune cells play a prominent role in the initiation, maintenance or resolution of the pathology, such inflammatory diseases and immune/autoimmune diseases. Examples of such diseases include multiple sclerosis, arthritis and vasculitis.

### SUMMARY OF THE INVENTION

The applicants have surprisingly discovered that (R)-carbidopa has useful SSAO/VAP-1 inhibitory activity, and that (R)-carbidopa has utility in the treatment or prevention of medical conditions wherein inhibition of VAP-1 activity is beneficial, such as inflammatory diseases, immune or autoimmune disorders and tumour growth.

Furthermore, the applicants have surprisingly discovered that (R)-carbidopa is effective in the treatment of pain, including inflammatory pain.

Furthermore, the applicants have discovered that (R)-carbidopa has surprising selectivity for SSAO/VAP-1 over the enzyme DOPA decarboxylase. This is especially surprising because the drug Lodosyn®, which comprises (S)-carbidopa is well-known as an inhibitor of DOPA decarboxylase. This advantageous selectivity is expected to be beneficial, particularly in the treatment of patients suffering from a disease or condition which benefits from inhibition of SSAO/VAP-1, but not DOPA decarboxylase.

Furthermore, the applicants have made available a combined preparation of (R)-carbidopa and a steroid. This combined preparation is expected to be useful in treatment or prevention of medical conditions wherein inhibition of VAP-1 activity is beneficial, such as inflammatory diseases, immune or autoimmune disorders and tumour growth.

Furthermore, the applicants have discovered that combined preparation of (R)-carbidopa and a steroid is surprisingly effective for the treatment of pain, including inflammatory pain.

### BRIEF DESCRIPTION OF THE FIGURES

Embodiments of the invention are described below, with reference to the accompanying drawings in which:

FIG. 1 shows the effects of (R)-carbidopa on CFA induced hyperalgesia in the rat at one hour and three hours post dose (left to right—vehicle; 3 mg/kg (R)-carbidopa; 10 mg/kg (R)-carbidopa; 30 mg/kg (R)-carbidopa; 10 mg/kg indomethacin);

FIG. 2 shows the effects of (R)-carbidopa and indomethacin on paw oedema in CFA-induced hyperalgesia in the rat at 3 hours post dose (left to right—vehicle/vehicle; 3 mg/kg (R)-carbidopa/vehicle; 10 mg/kg (R)-carbidopa/vehicle; 30 mg/kg (R)-carbidopa/vehicle; 10 mg/kg indomethacin);

FIG. 3 shows the effects of prednisolone on CFA-induced hyperalgesia in the rat at one hour and three hours post dose (left to right—vehicle/vehicle; 0.3 mg/kg prednisolone/vehicle; 1 mg/kg prednisolone/vehicle; 3 mg/kg prednisolone/vehicle; 10 mg/kg prednisolone/vehicle; 10 mg/kg indomethacin); and

FIG. 4 shows the effects of (R)-carbidopa and prednisolone on CFA-induced hyperalgesia in the rat at one hour and three hours post dose (left to right—vehicle/vehicle; 3 mg/kg (R)-carbidopa/vehicle; 10 mg/kg (R)-carbidopa/vehicle; 0.3 mg/kg prednisolone/vehicle; 3 mg/kg (R)-carbidopa/0.3 mg/kg prednisolone, 10 mg/kg (R)-carbidopa/0.3 mg/kg prednisolone).

### DETAILED DESCRIPTION OF THE INVENTION

### Definitions

As used herein, the terms “treatment,” “treating,” “treat” and the like, refer to obtaining a desired pharmacologic and/or physiologic effect. In the case of, for example, the treatment of pain, the effect can be prophylactic in terms of completely or partially preventing pain or a symptom thereof and/or can be therapeutic in terms of a partial or complete cure for pain and/or an adverse effect attributable to the disease. “Treatment,” as used herein, covers any treatment of pain in a mammal, particularly in a human, and includes: (a) preventing the disease from occurring in a subject which can be predisposed to the disease but has not yet been diagnosed as having it; (b) inhibiting the disease, i.e., arresting its development; and (c) relieving the disease, i.e., causing regression of the disease.

An “effective amount” of (R)-carbidopa and/or steroid refers to the amount of (R)-carbidopa and/or steroid that, when administered to a mammal or other subject for treating a disease or condition, is sufficient to effect such treatment for the disease or condition. The “effective amount” will vary depending on the steroid (if any), the disease and its severity and the age, weight, etc., of the subject to be treated. The therapeutic effect may be objective (i.e., measurable by some test or marker) or subjective (i.e., subject gives an indication of or feels an effect).

“Pharmaceutically acceptable” means being useful in preparing a pharmaceutical composition that is generally safe, non-toxic and neither biologically nor otherwise undesirable and includes being useful for veterinary use as well as human pharmaceutical use. Suitable pharmaceutically acceptable salts include, for example acid addition salts derived from inorganic or organic acids, such as hydrochlorides, hydrobromides, p-toluenesulphonates, phosphates, sulphates, perchlorates, acetates, trifluoroacetates, propionates, citrates, malonates, succinates, lactates, oxalates, tartrates and benzoates. For a review on salts, see Handbook of Pharmaceutical Salts: Properties, Selection, and Use by Stahl and Wermuth (Wiley-VCH, Weinheim, Germany, 2002). Pharmaceutically acceptable salts may also be formed with bases. Such salts include salts derived from inorganic or organic bases, for example alkali metal salts such as magnesium or calcium salts, and organic amine salts such as morpholine, piperidine, dimethylamine or diethylamine salts.

The term “pain” as used herein includes inflammatory pain. In an embodiment, the pain is inflammatory pain.

### Carbidopa

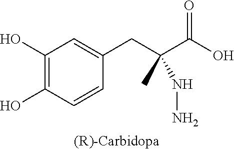 [SMILES: C[C@](Cc1ccc(O)c(O)c1)(NN)C(=O)O]

Carbidopa exists in the form of (R) and (S) enantiomers. Carbidopa is typically available as a mixture of the (R) and (S) enantiomers. Reference herein to “(R)-carbidopa” includes any composition or mixture comprising (R) carbidopa, including for example substantially pure (R)-carbidopa or mixtures of (R) and (S)-carbidopa, such as racemic mixtures.

In the practice of the present invention, it may be desirable to utilize a mixture that is predominantly (R)-carbidopa, such as a composition that is at least 60%, at least 70%, at least 80%, at least 90%, or at least 99% enantiomerically pure (R)-carbidopa. Use of a composition enriched in (R)-carbidopa can be desirable in order to take advantage of the benefit of the enzyme inhibitory selectivity described above.

### Steroid

The term “steroid” as used herein means any steroid suitable for use in the combined preparations according to the invention. The term “steroid” is also intended to encompass a combination of two or more steroids employed in the compositions and in the practice of the methods of the present invention.

Suitable steroids include glucocorticoids. Examples of glucocorticoid steroids include prednisolone, prednisone, methyl prednisolone, triamcinolone, dexamethasone, hydrocortisone, deflazacourt betamethasone and budenoside, or pharmaceutically acceptable salt thereof. Particularly preferred steroids include prednisolone, or a pharmaceutically acceptable salt thereof; and prednisone, or a pharmaceutically acceptable salt thereof.

### (R)-Carbidopa for the Treatment of Pain

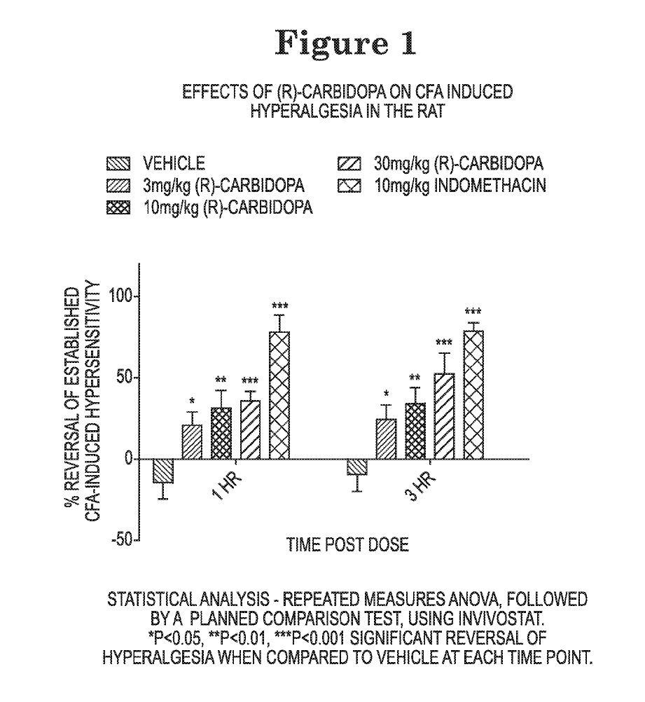

The applicants have discovered that (R)-carbidopa is surprisingly effective in the treatment of pain, including inflammatory pain. In vivo data demonstrating efficacy in a well-established model of pain is provided herein. See, for example, FIG. 1.

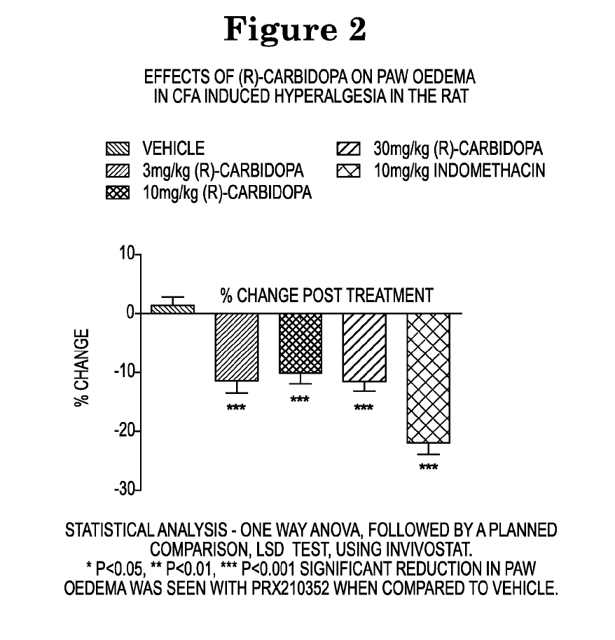

Furthermore, the applicant has found that (R)-carbidopa is surprisingly effective in the treatment of inflammation. In vivo data demonstrating efficacy in a well-established model of inflammation is provided herein. See, for example, FIG. 2.

Treatments of the present invention may provide any or all of the following: superior reduction of pain or inflammation; faster relief of pain or inflammation; increased compliance; decreased likelihood of addiction; reduced treatment-related side effects; the ability to reduce exposure to other therapeutic agents that exhibit dose-dependent treatment-related side effects; or any other perceptible therapeutic benefit.

### A Combined Preparation of (R)-Carbidopa and a Steroid

The applicants have found also that (R)-carbidopa in combination with a steroid is surprisingly effective in the treatment of pain. By surprisingly effective it is meant that (R)-carbidopa and the steroid together produce a therapeutic effect which is greater than the therapeutic effect of the (R)-carbidopa and the steroid when dosed individually. In an embodiment, (R)-carbidopa in combination with a steroid provides synergistic beneficial effects in the treatment of pain.

Therefore, in an embodiment, the present invention makes available a combined preparation comprising (R)-carbidopa or a hydrate or a pharmaceutically acceptable salt thereof, and a steroid or a pharmaceutically acceptable salt thereof. In an embodiment the steroid is a glucocorticoid. In an embodiment the steroid is a glucocorticoid selected from any one of prednisolone, prednisone, methyl prednisolone, triamcinolone, dexamethasone, hydrocortisone, deflazacourt, betamethasone and budenoside, or pharmaceutically acceptable salt thereof. In another embodiment, the steroid is a combination of two or more of any of the aforementioned steroids or salts thereof. In particular embodiments, the steroid is prednisolone, or a pharmaceutically acceptable salt thereof. In particular embodiments, the steroid is prednisone, or a pharmaceutically acceptable salt thereof.

Combinations of the present invention may provide any or all of the following: superior reduction of pain or inflammation; faster relief of pain or inflammation; increased compliance; decreased likelihood of addiction; reduced treatment-related side effects; the ability to reduce exposure to other therapeutic agents that exhibit dose-dependent treatment-related side effects; or any other perceptible therapeutic benefit.

In an embodiment the combined preparation comprises (R)-carbidopa or a hydrate or a pharmaceutically acceptable salt thereof, and prednisolone or pharmaceutically acceptable salt thereof.

In an embodiment the combined preparation comprises (R)-carbidopa or a hydrate or a pharmaceutically acceptable salt thereof, and prednisone or pharmaceutically acceptable salt thereof.

In an embodiment the combined preparation comprises (R)-carbidopa or a hydrate or a pharmaceutically acceptable salt thereof, and methyl prednisolone or pharmaceutically acceptable salt thereof.

In an embodiment the combined preparation comprises (R)-carbidopa or a hydrate or a pharmaceutically acceptable salt thereof, and triamcinolone or pharmaceutically acceptable salt thereof.

In an embodiment the combined preparation comprises (R)-carbidopa or a hydrate or a pharmaceutically acceptable salt thereof, and dexamethasone or pharmaceutically acceptable salt thereof.

In an embodiment the combined preparation comprises (R)-carbidopa or a hydrate or a pharmaceutically acceptable salt thereof, and hydrocortisone or pharmaceutically acceptable salt thereof.

In an embodiment the combined preparation comprises (R)-carbidopa or a hydrate or a pharmaceutically acceptable salt thereof, and deflazacourt or pharmaceutically acceptable salt thereof.

In an embodiment the combined preparation comprises (R)-carbidopa or a hydrate or a pharmaceutically acceptable salt thereof, and betamethasone or pharmaceutically acceptable salt thereof.

In an embodiment the combined preparation comprises (R)-carbidopa or a hydrate or a pharmaceutically acceptable salt thereof, and budenoside or pharmaceutically acceptable salt thereof.

### Compositions

A pharmaceutical composition containing the active ingredient, or active ingredients in the case of a combined preparation, may be in any suitable form, for example aqueous or non-aqueous solutions or suspensions, dispersible powders or granules, transdermal or transmucosal patches, creams, ointments or emulsions.

The pharmaceutical composition may be in the form of a sterile injectable aqueous or non-aqueous (e.g. oleaginous) solution or suspension. The sterile injectable preparation may also be in a sterile injectable solution or suspension in a non-toxic parenterally-acceptable diluent or solvent, for example as a solution in 1,3-butanediol. Among the acceptable vehicles and solvents that may be employed are water, phosphate buffer solution, Ringer's solution and isotonic sodium chloride solution. In addition, sterile, fixed oils are conventionally employed as a solvent or suspending medium. For this purpose, any bland fixed oil may be employed, including synthetic mono- or diglycerides. In addition, fatty acids such as oleic acid find use in the preparation of injectables. Suspensions may be formulated according to the known art using those suitable dispersing or wetting agents and suspending agents.

Aqueous suspensions contain the active ingredient, or active ingredients in the case of a combined preparation, in admixture with excipients suitable for the manufacture of aqueous suspensions. Such excipients are suspending agents, for example sodium carboxymethylcellulose, methylcellulose, hydroxypropylmethylcellulose, sodium alginate, polyvinyl-pyrrolidone, gum tragacanth and gum acacia; dispersing or wetting agents such as a naturally occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such a polyoxyethylene with partial esters derived from fatty acids and hexitol anhydrides, for example polyoxyethylene sorbitan monooleate. The aqueous suspensions may also contain one or more preservatives, for example ethyl or n-propyl p-hydroxybenzoate, one or more colouring agents, one or more flavouring agents, and one or more sweetening agents, such as sucrose or saccharin.

Non-aqueous (i.e. oily) suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are known.

The active agent may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols.

For topical delivery, transdermal and transmucosal patches, creams, ointments, jellies, solutions or suspensions may be employed. For sub-lingual delivery, fast dissolving tablet formulations may be used, as well as a number of the presentations described above. For oral administration, the drug may be administered as tablets, capsules or liquids.

Formulations may conveniently be presented in unit dosage form, e.g., tablets and sustained release capsules, and in liposomes, and may be prepared by any method known in the art of pharmacy. Pharmaceutical formulations are usually prepared by mixing the active substance, or a pharmaceutically acceptable salt thereof, with conventional pharmaceutically acceptable carriers, diluents or excipients. Examples of excipients are water, gelatin, gum arabicum, lactose, microcrystalline cellulose, starch, sodium starch glycolate, calcium hydrogen phosphate, magnesium stearate, talcum, colloidal silicon dioxide, and the like. Such formulations may also contain other pharmacologically active agents, and conventional additives, such as stabilizers, wetting agents, emulsifiers, flavouring agents, buffers, and the like. Usually, the amount of active compounds is between 0.1-95% by weight of the preparation, preferably between 0.2-20% by weight in preparations for parenteral use and more preferably between 1-50% by weight in preparations for oral administration. The formulations can be further prepared by known methods such as granulation, compression, microencapsulation, spray coating, etc. The formulations may be prepared by conventional methods in the dosage form of tablets, capsules, granules, powders, syrups, suspensions, suppositories or injections. Liquid formulations may be prepared by dissolving or suspending the active substance in water or other suitable vehicles. Tablets and granules may be coated in a conventional manner. To maintain therapeutically effective plasma concentrations for extended periods of time, compounds of the invention may be incorporated into slow release formulations.

The dose level and frequency of dosage of the specific compound will vary depending on a variety of factors including the potency of the specific compound employed, the metabolic stability and length of action of that compound, the patient's age, body weight, general health, sex, diet, mode and time of administration, rate of excretion, drug combination, the severity of the condition to be treated, and the patient undergoing therapy. The daily dosage may, for example, range from about 0.001 mg to about 100 mg per kilo of body weight, administered singly or multiply in doses, e.g. from about 0.01 mg to about 25 mg each. Such a dosage may be given orally or parenterally. Multiple doses may be administered over a period of time, such as at least a week, a month, several months, a year, or several years, or throughout the course of the condition. The frequency of dosage may be at least once per month, once per week, or once per day.

### Combined Preparations

The components of a combined preparation according to the present invention may be for simultaneous, separate, or sequential use.

The term “combined preparation” as used herein refers to a “kit of parts” in the sense that the combination components of (a) (R)-carbidopa and (b) a steroid can be dosed independently or by use of different fixed combinations with distinguished amounts of the combination components (a) and (b). The components can be administered simultaneously or one after the other. If the components are administered one after the other, preferably the time interval between administrations is chosen such that the effect on the treated disorder or disease in the combined use of the components is greater than the effect which would be obtained by use of only any one of the combination components (a) and (b).

The components of the combined preparation may be present in one combined unit dosage form, or as a first unit dosage form of component (a) and a separate, second unit dosage form of component (b). The ratio of the total amounts of the combination component (a) to the combination component (b) to be administered in the combined preparation can be varied, for example in order to cope with the needs of a patient sub-population to be treated, or the needs of the single patient, which can be due, for example, to the particular disease, age, sex, or body weight of the patients.

Preferably, there is at least one beneficial effect, for example an enhancing of the effect of the (R)-carbidopa inhibitor, or a mutual enhancing of the effect of the combination components (a) and (b), for example a more than additive effect, additional advantageous effects, fewer side effects, less toxicity, or a combined therapeutic effect compared with a non-effective dosage of one or both of the combination components (a) and (b), and very preferably a synergism of the combination components (a) and (b).

The (R)-carbidopa and the steroid may be administered sequentially to the subject, i.e. the (R)-carbidopa may be administered before, with, or after the steroid.

The (R)-carbidopa and the steroid may be administered to the subject within 96 hours, 72 hours, 48 hours, 24 hours, or 12 hours, of each other.

Alternatively, the (R)-carbidopa and the steroid may be co-administered to the subject, for example as a composition comprising the VAP-1 inhibitor and the steroid, or by simultaneous administration of separate doses of the (R)-carbidopa and the steroid.

According to some embodiments, a plurality of doses of the (R)-carbidopa, and/or a plurality of doses of the steroid, is administered to the subject.

According to some embodiments, a dose of the (R)-carbidopa is administered before, with, or after each administration of two or more doses of the steroid.

For example, a dose of (R)-carbidopa may be administered within 96 hours, 72 hours, 48 hours, 24 hours, or 12 hours, of each administration of two or more doses of the steroid.

The choice of appropriate dosages of the components used in combination therapy according to the present invention can be determined and optimized by the skilled person, for example, by observation of the patient, including the patient's overall health, and the response to the combination therapy. Optimization, for example, may be necessary if it is determined that a patient is not exhibiting the desired therapeutic effect or conversely, if the patient is experiencing undesirable or adverse side effects that are too many in number or are of a troublesome severity.

The doses of the components used in combination therapy according to the invention should be chosen to provide a therapeutically effective amount of the components in combination. An “effective amount” of the combination therapy is an amount that results in a reduction of at least one pathological parameter associated with pain. For example, in some embodiments, an effective amount of the combination therapy is an amount that is effective to achieve a reduction of at least about 10%, 20%, 30%, 40%, 50%, 60%, 70%, 80%, or 90%, in the parameter, compared to the expected reduction in the parameter associated with the pain without the combination therapy. For example, the parameter may be a score resulting from an assessment under the Western Ontario and McMaster Universities Arthritis Index (WOMAC), for example for pain during walking, using stairs, in bed, sitting or lying, and standing, or daily activity, physical function or stiffness scores. Alternatively the parameter may be a score from an assessment on the Visual Analogue Scale (VAS), Pain Intensity (PI) Scale, Wong-Baker FACES Pain Rating Scale, 0-10 Numeric Pain Rating Scale, Verbal Pain Intensity Scale or Descriptor Differential Scale.

According to the invention, combination treatment may be employed to increase the therapeutic effect of the (R)-carbidopa or steroid, compared with the effect of the (R)-carbidopa or steroid as a monotherapy, or to decrease the doses of the individual components in the resulting combinations while preventing or further reducing the risk of unwanted or harmful side effects of the individual components.

A typically prescribed dose range for a steroid as a monotherapy, in particular a glucocorticoid such as prednisone or prednisolone, is 0.3-1 mg/kg/day (suitably 0.7 or 0.75 mg/kg/day), or 0.3 mg/kg/day to 10 mg/kg/week, in humans.

A typically prescribed dose range for (R)-carbidopa as a monotherapy in humans is 20-200 mg/day, typically 30 mg/day or 75 mg/day.

In one embodiment, the (R)-carbidopa and the steroid are each prescribed at a dose that is within a typically prescribed dose range for each compound as a monotherapy. The compounds may be prescribed as separate dosages or as a combination dosage. Such combinations provide increased efficacy compared with the effect of either compound as a monotherapy.

In another embodiment, the (R)-carbidopa and the steroid are each prescribed at a dose that is below a typically prescribed dose for each component as a monotherapy, but at doses that have therapeutic efficacy in combination. The components may be prescribed as separate dosages or as a combination dosage. The dosages of the components in combination may be selected to provide a similar level of therapeutic efficacy as the (R)-carbidopa or the steroid as a monotherapy, but with the advantage that the lower doses of the (R)-carbidopa and/or the steroid reduce the risk of adverse side effects compared to the prescribed dosages of each compound as a monotherapy.

In another embodiment, the prescribed dosage of the (R)-carbidopa is within a typically prescribed dose range for monotherapy, and the steroid is prescribed at a dosage that is below a typically prescribed dose for monotherapy.

In a further embodiment, the prescribed dosage of the (R)-carbidopa is below a typically prescribed dose for monotherapy, and the steroid is prescribed at a dosage that is within a typically prescribed dose range for monotherapy.

Preferred dosages below the typically prescribed dose for monotherapy are doses that are up to 50%, or up to 25%, of the typically prescribed dose.

When administered in separate dosages, the (R)-carbidopa and the steroid may be administered substantially simultaneously (for example, within about 60 minutes, about 50 minutes, about 40 minutes, about 30 minutes, about 20 minutes, about 10 minutes, about 5 minutes, or about 1 minute of each other) or separated in time by about 1 hour, about 2 hours, about 4 hours, about 6 hours, about 10 hours, about 12 hours, about 24 hours, about 36 hours, about 72 hours, or about 96 hours, or more.

The skilled person will be able to determine, and optimise, a suitable time course for sequential administration, depending on the particular combination of the (R)-carbidopa and the steroid. The time course is preferably selected such that there is at least one beneficial effect, for example an enhancing of the effect of the (R)-carbidopa or the steroid, or a mutual enhancing of the effect of the combination components, for example a more than additive effect, additional advantageous effects, fewer side effects, less toxicity, or a combined therapeutic effect compared with a non-effective dosage of one or both of the combination components, and very preferably a synergism of the combination components.

It will be appreciated that the optimum time course will depend on factors such as the time taken for the peak plasma concentration of the compound to be reached after administration, and the elimination half-life of each compound. Preferably the time difference is less than the half-life of the first component to be administered.

The skilled person will also be able to determine appropriate timing for administration. In certain embodiments, the (R)-carbidopa may be administered in the morning, and the steroid administered at least once later in the day. In other embodiments, the (R)-carbidopa and the steroid may be administered at substantially the same time.

The subject may receive doses of the (R)-carbidopa inhibitor and the steroid over a period of weeks, months, or years. For example, 1 week, 2 weeks, 3 weeks, 1 month, 2 months, 3 months, 4 months, 5 months, 6 months, 7 months, 8 months, 9 months, 10 months, 11 months, 1 year, 2 years, 3 years, 4 years, 5 years, or more.

In general, the components of a combination of the invention may be administered by known means, in any suitable formulation, by any suitable route. Suitable routes of administration may include by oral, rectal, nasal, topical (including buccal and sublingual), sublingual, transdermal, intrathecal, transmucosal or parenteral (including subcutaneous, intramuscular, intravenous and intradermal) administration. In some embodiments, the (R)-carbidopa inhibitor and the steroid are administered orally.

Suitable pharmaceutical compositions and dosage forms may be prepared using conventional methods known to those in the field of pharmaceutical formulation and described in the relevant texts and literature, for example, in Remington: The Science and Practice of Pharmacy (Easton, Pa.: Mack Publishing Co., 1995).

It is especially advantageous to formulate combined preparations of the invention in unit dosage form for ease of administration and uniformity of dosage. The term “unit dosage forms” as used herein refers to physically discrete units suited as unitary dosages for the individuals to be treated. That is, the compositions are formulated into discrete dosage units each containing a predetermined, “unit dosage” quantity of an active agent calculated to produce the desired therapeutic effect in association with the required pharmaceutical carrier. The specifications of unit dosage forms of the invention are dependent on the unique characteristics of the active agent to be delivered. Dosages can further be determined by reference to the usual dose and manner of administration of the ingredients. It should be noted that, in some cases, two or more individual dosage units in combination provide a therapeutically effective amount of the active agent, for example, two tablets or capsules taken together may provide a therapeutically effective dosage, such that the unit dosage in each tablet or capsule is approximately 50% of the therapeutically effective amount.

Preparations according to the invention for parenteral administration include sterile aqueous and non-aqueous solutions, suspensions, and emulsions. Injectable aqueous solutions contain the active agent in water-soluble form. Examples of non-aqueous solvents or vehicles include fatty oils, such as olive oil and corn oil, synthetic fatty acid esters, such as ethyl oleate or triglycerides, low molecular weight alcohols such as propylene glycol, synthetic hydrophilic polymers such as polyethylene glycol, liposomes, and the like. Parenteral formulations may also contain adjuvants such as solubilizers, preservatives, wetting agents, emulsifiers, dispersants, and stabilizers, and aqueous suspensions may contain substances that increase the viscosity of the suspension, such as sodium carboxymethyl cellulose, sorbitol, and dextran. Injectable formulations may be rendered sterile by incorporation of a sterilizing agent, filtration through a bacteria-retaining filter, irradiation, or heat. They can also be manufactured using a sterile injectable medium. The active agent may also be in dried, e.g., lyophilized, form that may be rehydrated with a suitable vehicle immediately prior to administration via injection.

In addition to the formulations described previously, the active agent may be formulated as a depot preparation for controlled release of the active agent, preferably sustained release over an extended time period. These sustained release dosage forms are generally administered by implantation (for example, subcutaneously or intramuscularly or by intramuscular injection).

Combined preparations of the invention may be packaged with instructions for administration of the components on the combination. The instructions may be recorded on a suitable recording medium or substrate. For example, the instructions may be printed on a substrate, such as paper or plastic. The instructions may be present as a package insert, in the labeling of the container or components thereof (i.e., associated with the packaging or sub-packaging). In other embodiments, the instructions are present as an electronic storage data file present on a suitable computer readable storage medium, for example, CD-ROM, diskette. Some or all components of the combined preparation may be packaged in suitable packaging to maintain sterility.

### Biological Data

### Example 1

### In Vitro Determination of Human VAP-1 (SSAO) Inhibition

This assay is performed at room temperature with purified recombinantly expressed human VAP-1 (SSAO). Enzyme was prepared essentially as described in Ohman et al. (Protein Expression and Purification 46 (2006) 321-331). The enzyme activity is assayed with benzylamine as substrate by utilizing the production of hydrogen peroxide in a horseradish peroxidise (HRP) coupled reaction. Briefly, test compounds are dissolved in dimethyl sulfoxide (DMSO) to a concentration of 10 mM. Dose-response measurements are assayed by either creating 1:10 serial dilutions in DMSO to produce a 7 point curve or by making 1:3 serial dilutions in DMSO to produce 11 point curves. The top concentrations are adjusted depending on the potency of the compounds and subsequent dilution in reaction buffer yielded a final DMSO concentration ≤2%.

In a horseradish peroxidise (HRP) coupled reaction, hydrogen peroxide oxidation of 10-acetyl-3,7-dihydroxyphenoxazine produces resorufin, which is a highly fluorescent compound (Zhout and Panchuk-Voloshina. Analytical Biochemistry 253 (1997) 169-174; AmplexR Red Hydrogen Peroxide/peroxidise Assay kit, Invitrogen A22188). Enzyme and compounds in 50 mM sodium phosphate, pH 7.4 are set to pre-incubate in flat-bottomed microtiter plates for approximately 15 minutes before initiating the reaction by addition of a mixture of HRP, benzylamine and Amplex reagent. Benzylamine concentration is fixed at a concentration corresponding to the Michaelis constant, determined using standard procedures. Fluorescence intensity is then measured at several time points during 1-2 hours, exciting at 544 nm and reading the emission at 590 nm. For the human SSAO assay final concentrations of the reagents in the assay wells are: SSAO enzyme 1 mg/ml, benzylamine 100 μM, Amplex reagent 20 μM, HRP 0.1 U/mL and varying concentrations of test compound. The inhibition is measured as % decrease of the signal compared to a control without inhibitor (only diluted DMSO). The background signal from a sample containing no SSAO enzyme is subtracted from all data points. Data is fitted to a four parameter logistic model and IC50 values are calculated, for example by using the GraphPad Prism 4 or XLfit 4 programs.

Using this assay, the IC50s for inhibition of human VAP-1 by (S)-Carbidopa and (R)-Carbidopa were 142 nM and 148 nM, respectively.

### Example 2

### In Vitro Determination of Human Aromatic L-Amino Acid Decarboxylase (DOPA Decarboxylase) Inhibition

The enzyme reaction of human DOPA decarboxylase was carried out according to the manufacture instructions (R&D systems cat#3564-DC). The assay measured the ability of recombinant human DOPA decarboxylase to convert L-Dopa to dopamine. The dopamine product was measured by its absorbance at 340 nm after derivatization with trinitrobenzene sulphuric acid. Briefly, the reaction was carried out in 50 mM HEPES buffer containing 100 mM NaCl pH 7.2 for 30 min and stopped by inactivation of the enzyme at 95° C. for 2 min. The inhibitory assay was performed by measuring the decarboxylase activity of DOPA decarboxylase with 0.8 μg enzyme, 500 μM L-Dopa, and 100 μM pyridoxal phosphate in the presence or absence of a fixed amount of each compound. Test compounds were pre-incubated for 60 min with DOPA decarboxylase before initiating the assay with the addition of the substrate L-DOPA. Dose response curves were generated to determine the concentration required to inhibit 50% of decarboxylase activity (IC50). Compounds were evaluated in duplicate at 7 concentrations. IC50 values were derived by nonlinear regression analysis.

Using this assay, the IC50 for inhibition of human aromatic L-amino acid decarboxylase by (S)-Carbidopa was 190 nM, whereas (R)-Carbidopa did not inhibit human aromatic L-amino acid decarboxylase up to the maximum concentration tested (3 uM).

### Example 3

Effect of (R)-Carbidopa, Both Alone and in Combination with Prednisolone on CFA (Complete Freunds Adjuvant) Induced Hypersensitivity in Rat

Assessment of the anti-hyperalgesic properties of (R)-Carbidopa was determined through measurement of weight bearing following CFA induced hypersensitivity. Naive rats distribute their body weight equally between the two hind paws. However, when the injected (left) hind paw is painful, the weight is re-distributed so that less weight is put on the affected paw (decrease in weight bearing on injured paw). Weight bearing through each hind limb was measured using a rat incapacitance tester (Linton Instruments, UK). Rats were placed in the incapacitance tester with the hind paws on separate sensors and the average force exerted by both hind limbs was recorded over 4 seconds. The injection of CFA also induces an oedema that can be assessed by paw volume; this is measured using a plethysmometer. The rat's hind paw is placed into the cylinder containing a solution and the volume of displaced liquid determines the paw volume.

Naive male, Sprague Dawley rats were acclimatised with food and water available ad libitum. Habituation to the incapacitance tester was performed. Baseline weight bearing and paw volume recordings were taken prior to induction of insult. Inflammatory hypersensitivity was induced by intraplantar injection of CFA (100 μl of 1 mg/ml solution) into the left hind paw. A pre-treatment weight bearing and paw volume measurement was taken to assess hypersensitivity 23 hours post-CFA. Animals were then ranked and randomised according to CFA window in a Latin square design.

In Part A, animals were treated with either Vehicle (5% DMSO, 0.5% Hydroxypropyl methylcellulose (HPMC) in water), (R)-Carbidopa 3, 10 & 30 mg/kg or Indomethacin 10 mg/kg (10 mL/kg dose volume) 24 hours post CFA. Weight bearing was measured at 1 and 3 hours post treatment and oedema was measured 3 hours post treatment.

In Part B: Animals were treated with either Vehicle (1% Methylcellulose (MC) in water), Prednisolone 0.3, 1, 3 & 10 mg/kg, or Indomethacin 10 mg/kg (5 mL/kg dose volume) 24 hours post CFA. Weight bearing was measured at 1 and 3 hours post treatment.

In Part C, animals were treated twice, once with either Vehicle (5% DMSO 0.5% HPMC) or (R)-Carbidopa 3 & 10 mg/kg p.o. (10 mL/kg dose volume) and then with either Vehicle (1% MC) or Prednisolone 0.3 mg/kg p.o. (5 mL/kg dose volume) 24 hours post CFA. Weight bearing was measured at 1 and 3 hours post treatment and oedema was measured 3 hours post treatment.

Data were analysed by comparing treatment groups to the vehicle control group at each time point.

Weight bearing (g) readings were taken for both right and left hind paws and the difference calculated. Data is expressed as % reversal of the hypersensitivity to pain. Paw Volume (mL) readings were taken for the left hind paws. Data are expressed as % reversal of the oedema. Calculation: (post dose reading−pre dose reading)/(naïve reading−pre dose reading)×100, where naive weight bearing difference−pre dose weight bearing difference is defined as the CFA window to be reversed. Statistical analysis was conducted by means of repeated measures ANOVA followed by Planned comparison test using InVivoStat (invivostat.co.uk), (p<0.05 considered significant).

### Results

Intraplantar injection of CFA induced hypersensitivity as detected by a shift in weight bearing between injured and non-injured hind paws 24 hours post dose. CFA also induced a marked oedema in the injected paw in both studies. In line with previous studies, indomethacin (10 mg/kg) produced a marked reversal of the hypersensitivity measured using weight bearing. Indomethacin also showed a significant reduction on paw oedema.

Part A: (R)-Carbidopa (3-30 mg/kg) alone dose-dependently inhibited the hypersensitivity response (see FIG. 1) and showed a significant reduction of paw volume (see FIG. 2).

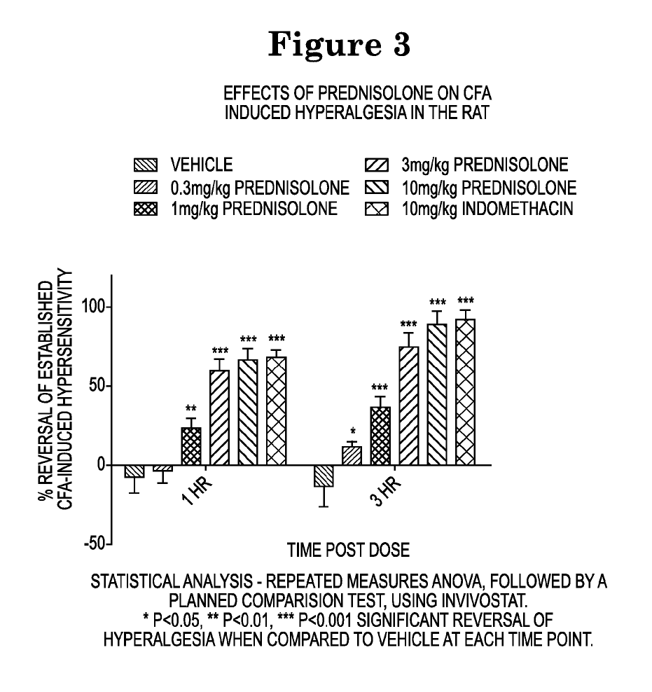

Part B: Prednisolone (0.3-10 mg/kg) alone dose-dependently inhibited the hypersensitivity response (see FIG. 3).

Part C: Minimally/moderately effective doses of (R)-Carbidopa (3 & 10 mg/kg) and prednisolone (0.3 mg/kg) were selected to be administered in combination in order to evaluate potential synergistic effects.

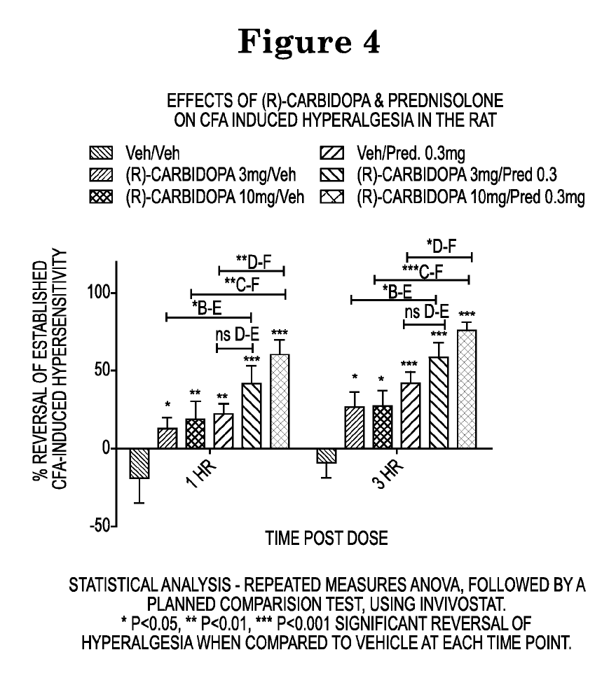

Co-dosing prednisolone (0.3 mg/kg) with (R)-carbidopa had the same analgesic effect as 3 mg/kg prednisolone alone, suggesting that steroid dosing can be reduced by more than 10-fold when co-dosed with (R)-carbidopa (see FIG. 4).

The results also show evidence of synergy between prednisolone and (R)-carbidopa (see FIG. 4).

Synergy can be calculated according to the methods taught in references [1] and [2]:
[1] Webb J L, Effect of more than one inhibitor. Enzyme and metabolic inhibitors. 1. New York: Academic Press; 1963, p. 66-79 (488-512)
[2] Greco W R, Bravo G, and Parsons J C (1995) The search for synergy: a critical review from a response surface perspective. Pharmacol Rev 47: 331-385.

1.-2. (canceled)

3. A method for the treatment of a disease or condition selected from pain, inflammation, an inflammatory disease, an immune or an autoimmune disorder and tumour growth, which comprises administering to a subject suffering from such a disease an effective amount of (R)-carbidopa or a hydrate or a pharmaceutically acceptable salt thereof.

4. (canceled)

5. A method according to claim 3, wherein the (R)-carbidopa is at least 60%, preferably at least 70%, more preferably at least 80%, yet more preferably at least 90%, or even more preferably at least 99% enantiomerically pure (R)-carbidopa.

6. A method according to claim 3, wherein the disease or condition is pain.

7. A method according to claim 6, wherein the disease or condition is inflammatory pain.

8. A method according to claim 3, wherein the disease or condition is inflammation.

9. A method according to claim 3, wherein the inflammation or inflammatory disease or immune or autoimmune disorder is lupus (systemic lupus erythematosus), arthritis (including rheumatoid arthritis, juvenile rheumatoid arthritis, osteoarthritis and psoriatic arthritis), synovitis, vasculitis, a condition associated with inflammation of the bowel (including Crohn's disease, ulcerative colitis, inflammatory bowel disease and irritable bowel syndrome), atherosclerosis, multiple sclerosis, Alzheimer's disease, vascular dementia, a pulmonary inflammatory disease (including asthma, chronic obstructive pulmonary disease and acute respiratory distress syndrome), a fibrotic disease (including idiopathic pulmonary fibrosis, cardiac fibrosis and systemic sclerosis (scleroderma)), an inflammatory disease of the skin (including contact dermatitis, atopic dermatitis and psoriasis), systemic inflammatory response syndrome, sepsis, an inflammatory and/or autoimmune condition of the liver (including autoimmune hepatitis, primary biliary cirrhosis, alcoholic liver disease, sclerosing cholangitis, and autoimmune cholangitis), diabetes (type I or II) and/or the complications thereof, chronic heart failure, congestive heart failure, an ischernic disease (including stroke and ischemia-reperfusion injury) or myocardial infarction and/or the complications thereof.

10. A method according to claim 3, wherein the inflammatory or autoimmune disease is rheumatoid arthritis, chronic obstructive pulmonary disease or atopic dermatitis.

11. A method according to claim 3, wherein the inflammatory or autoimmune disease is inflammatory bowel disease, osteoarthritis, chronic obstructive pulmonary disease, fibrosis, or lupus (systemic lupus erythematosus).

12. A combined preparation comprising (R)-carbidopa or a hydrate or a pharmaceutically acceptable salt thereof, and a steroid or a pharmaceutically acceptable salt thereof.

13. A combined preparation according to claim 12 wherein the steroid is a glucocorticoid.

14. A combined preparation according to claim 13 wherein the steroid is a glucocorticoid selected from any one of prednisolone, prednisone, methyl prednisolone, triamcinolone, dexamethasone, hydrocortisone, deflazacourt, betamethasone and budenoside, or pharmaceutically acceptable salt thereof.

15. A combined preparation according to claim 12 wherein the (R)-carbidopa is at least 60%, preferably at least 70%, more preferably at least 80%, yet more preferably at least 90%, or even more preferably at least 99% enantiomerically pure (R)-carbidopa.

16.-18. (canceled)

19. A method for the treatment of a disease or condition selected from pain, inflammation, an inflammatory disease, an immune or an autoimmune disorder and tumour growth, which comprises administering to a subject suffering from such a disease an effective amount of a combined preparation according to claim 12.

20. (canceled)

21. A method according to claim 19, wherein the disease or condition is pain.

22. A method according to claim 21, wherein the disease or condition is inflammatory pain.

23. A method according to claim 19, wherein the disease or condition is inflammation.

24. A method according to claim 19, wherein the inflammation or inflammatory disease or immune or autoimmune disorder is lupus (systemic lupus erythematosus), arthritis (including rheumatoid arthritis, juvenile rheumatoid arthritis, osteoarthritis and psoriatic arthritis), synovitis, vasculitis, a condition associated with inflammation of the bowel (including Crohn's disease, ulcerative colitis, inflammatory bowel disease and irritable bowel syndrome), atherosclerosis, multiple sclerosis, Alzheimer's disease, vascular dementia, a pulmonary inflammatory disease (including asthma, chronic obstructive pulmonary disease and acute respiratory distress syndrome), a fibrotic disease (including idiopathic pulmonary fibrosis, cardiac fibrosis and systemic sclerosis (scleroderma)), an inflammatory disease of the skin (including contact dermatitis, atopic dermatitis and psoriasis), systemic inflammatory response syndrome, sepsis, an inflammatory and/or autoimmune condition of the liver (including autoimmune hepatitis, primary biliary cirrhosis, alcoholic liver disease, sclerosing cholangitis, and autoimmune cholangitis), diabetes (type I or II) and/or the complications thereof, chronic heart failure, congestive heart failure, an ischemic disease (including stroke and ischemia-reperfusion injury) or myocardial infarction and/or the complications thereof.

25. A method according to claim 19, wherein the inflammatory or autoimmune disease is rheumatoid arthritis, chronic obstructive pulmonary disease or atopic dermatitis.

26. A method according to claim 19, wherein the inflammatory or autoimmune disease is inflammatory bowel disease, osteoarthritis, chronic obstructive pulmonary disease, fibrosis, or lupus (systemic lupus erythematosus).

27. A method of treatment according to claim 3, wherein the treatment is treatment in a human subject.
